(19) United States
(12) Patent Application Publication
(10) Pub. No.: US 2017/0217853 A1
(43) Pub. Date: Aug. 3, 2017

(54) METHOD AND SYSTEM FOR PRODUCING AN OLEFIN

(71) Applicant: AGENCY FOR SCIENCE, TECHNOLOGY AND RESEARCH, Singapore (SG)

(72) Inventors: Su Seong LEE, Singapore (SG); Jian Liang CHEONG, Singapore (SG)

(21) Appl. No.: 15/515,131

(22) PCT Filed: Oct. 5, 2015
(86) PCT No.: PCT/SG2015/050372

§ 371 Date: Mar. 28, 2017

(30) Foreign Application Priority Data
Oct. 3, 2014 (SG) 10201406353Y

(51) Int. Cl. C07C 1/20; B01J 21/06; C07C 45/71; B01J 23/72; B01J 23/10; B01J 23/06; B01J 8/04; B01J 21/10

(52) U.S. Cl. CPC C07C 1/20; B01J 8/04; B01J 21/06; B01J 21/066; B01J 21/10; B01J 23/72; B01J 23/10; B01J 23/06; C07C 45/71; C07C 2521/06; C07C 2523/72; C07C 2521/10; C07C 2523/10; C07C 2523/06; B01J 2208/025

(57) ABSTRACT

There is provided a method for producing an optionally substituted olefin, comprising the steps of: dehydrogenating an optionally substituted alcohol in a first reaction zone comprising a first catalyst supported on a porous silica-based particle to form an optionally substituted carbonyl at a first set of reaction conditions; converting the optionally substituted alcohol and the optionally substituted carbonyl from the first reaction zone in a second reaction zone at a second set of reaction conditions that is different to the first set of reaction conditions and is selected to form the optionally substituted olefin, wherein the second reaction zone comprises a second catalyst supported on a porous silica-based particle. There is also provided a system for producing the optionally substituted olefin.

### TECHNICAL FIELD

The present invention generally relates to the preparation of chemical compounds, and more specifically, the preparation of optionally substituted olefins.

### BACKGROUND ART

Developing chemical reactions or processes to transform renewable carbon-neutral feedstock such as biomass into valuable chemical commodities is one of the most pressing issues in the 21st century. Such reactions will reduce our dependence on fossil fuels, ultimately bringing about benefits to the environment and the economy.

1,3-butadiene (BD) is a high-value chemical monomer used for the production of commercial polymers such as styrene-butadiene rubbers. BD can be converted from ethanol either directly or via a two or more step process. In recent times, BD is extracted from oil via steam cracking of petroleum gas or naphtha. However, with the rising prices of crude oil and decreasing prices of chemicals derived from cheap biomass, reactions like ethanol-to-butadiene conversion have come to the fore again in recent years.

Several catalyst systems have been used in the conversion of ethanol to BD. However, percentage conversion of ethanol and selectivity of BD have not been satisfactory for practical industrial application.

There is therefore a need to provide a method and system that overcomes, or at least ameliorates, one or more of the disadvantages described above.

### SUMMARY OF INVENTION

According to a first aspect, there is provided a method for producing an optionally substituted olefin, comprising the steps of: dehydrogenating an optionally substituted alcohol in a first reaction zone comprising a first catalyst supported on a porous silica-based particle to form an optionally substituted carbonyl at a first set of reaction conditions; converting the optionally substituted alcohol and the optionally substituted carbonyl from the first reaction zone in a second reaction zone at a second set of reaction conditions that is different to the first set of reaction conditions and is selected to form the optionally substituted olefin, wherein the second reaction zone comprises a second catalyst supported on a porous silica-based particle.

According to a second aspect, there is provided a system for producing an optionally substituted olefin, the system comprising: a first reaction zone having a first catalyst for dehydrogenating an optionally substituted alcohol to form an optionally substituted carbonyl; and a second reaction zone having a second catalyst for converting the optionally substituted alcohol and the optionally substituted carbonyl from the first reaction zone to form the optionally substituted olefin; wherein the first and second catalysts are supported on a porous silica-based particle.

### Definitions

The following words and terms used herein shall have the meaning indicated:

The term “foam”, as used herein, refers to a particle, consisting of mesopores that are interconnected by uniform pores of smaller sizes (known as window pores) as compared to the mesopores, thereby creating a three-dimensional porous system or network. The pore size of the mesopore within such a foam particle tends to be in the range of 5 nm to 50 nm and the size of the foam particle tends to be in the range of at least 1 um or 1 um to 100 um. The size of the window pore may be at least 2 nm or in the range of 2 nm to 100 nm. The ratio of the size of the mesopores and the size of the window pores may be between 12:1 and 1.1:1 or some other ratio as long as the mesopores have a pore size larger than the window pores.

The term “olefin”, which may be used interchangeably with the term “alkene”, as used herein means any unsaturated hydrocarbon containing one or more pairs of carbon atoms linked by a double bond. The olefins may be cyclic or acyclic (aliphatic) olefins, in which the double bond is located between carbon atoms forming part of a cyclic (closed-ring) or of an open-chain grouping, respectively. “Olefin” may also refer to monoolefins, diolefins, triolefins, etc., in which the number of double bonds per molecule is, respectively, one, two, three, or some other number. The orientation about each double bond may independently be of E, Z, cis or trans stereochemistry where applicable.

The term “diene” as used herein, refers to an unsaturated hydrocarbon that contains two carbon double bonds, particularly having at least 4 carbon atoms, such as but not limited to, a C₄-C₂₀ diene or a diene having any number of carbon atoms falling within this range. Accordingly, “dienes” may be used interchangeably with the term “diolefins”. Likewise, the orientation about each double bond may independently be of E, Z, cis or trans stereochemistry where applicable.

The term “optionally substituted” as used herein means the group to which this term refers may be unsubstituted, or may be substituted with one or more groups independently selected from —C₁-C₂₀-alkyl, —C₁-C₂₀-alkenyl, —C₁-C₂₀-alkynyl, —C₃-C₂₀-cycloalkyl, —C₅-C₂₀-cycloalkenyl, —C₅-C₂₀-heterocycloalkyl having 1 to 5 hetero atoms selected from N, O and S in the ring, halo, —C₁-C₂₀-haloalkyl.

“Alkyl” as a group or part of a group refers to a straight or branched aliphatic hydrocarbon group, particularly but not limited to, at least one carbon atom, or a C₁-C₂₀ alkyl, a C₁-C₁₀ alkyl, a C₁-C₆ alkyl or any number of carbon atoms falling within these ranges. Examples of suitable straight and branched C₁-C₆ alkyl substituents include methyl, ethyl, n-propyl, 2-propyl, n-butyl, sec-butyl, t-butyl, hexyl, and the like. The group may be a terminal group or a bridging group.

“Alkenyl” as a group or part of a group denotes an aliphatic hydrocarbon group containing at least one carbon-carbon double bond and which may be straight or branched having but not limited to, at least 2 carbon atoms, 2-20 carbon atoms, 2-10 carbon atoms, 2-6 carbon atoms, or any number of carbons falling within these ranges, in the normal chain. The group may contain a plurality of double bonds in the normal chain and the orientation about each is independently E, Z, cis or trans where applicable. Exemplary alkenyl groups include, but are not limited to, ethenyl, propenyl, butenyl, pentenyl, hexenyl, heptenyl, octenyl and nonenyl. The group may be a terminal group or a bridging group.

The term “alkynyl group” as used herein includes within its meaning straight or branched chain unsaturated aliphatic hydrocarbon groups having but not limited to, at least 2 carbon atoms or 2 to 20 carbon atoms, and having at least one triple bond anywhere in the carbon chain. Examples of alkynyl groups include but are not limited to ethynyl, 1-propynyl, 1-butynyl, 2-butynyl, 1-methyl-2-butynyl, 3-methyl-1-butynyl, 1-pentynyl, 1-hexynyl, methylpentynyl, 1-heptynyl, 2-heptynyl, 1-octynyl, 2-octynyl, 1-nonyl, 1-decynyl, and the like.

“Cycloalkyl” refers to a saturated monocyclic or fused or spiro polycyclic, carbocycle containing at least 3 carbons atoms or from 3 to 20 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. A cycloalkyl group typically is a C₃-C₂₀ alkyl group. The group may be a terminal group or a bridging group.

The term “cycloalkenyl” as used herein, refers to cyclic unsaturated aliphatic groups and includes within its meaning monocyclic, bicyclic, polycyclic or fused polycyclic hydrocarbon radicals having at least 3 carbon atoms or from 3 to 20 carbon atoms and having at least one double bond, of either E, Z, cis or trans stereochemistry where applicable, anywhere in the alkyl chain. Examples of cycloalkenyl groups include but are not limited to cyclopropenyl, cyclopentenyl, cyclohexenyl, and the like.

The term “heterocycloalkyl” as used herein, includes within its meaning monovalent (“heterocycloalkyl”) and divalent (“heterocycloalkylene”), saturated, monocyclic, bicyclic, polycyclic or fused hydrocarbon radicals having at least 3 carbon atoms or from 3 to 20 ring atoms wherein 1 to 5 ring atoms are heteroatoms selected from O, N, NH, or S. Examples include pyrrolidinyl, piperidinyl, quinuclidinyl, azetidinyl, morpholinyl, tetrahydrothiophenyl, tetrahydrofuranyl, tetrahydropyranyl, and the like.

The term “halo” or variants such as “halide” or “halogen” as used herein refer to fluorine, chlorine, bromine and iodine.

“Haloalkyl” refers to an alkyl group as defined herein in which one or more of the hydrogen atoms has been replaced with a halogen atom selected from the group consisting of fluorine, chlorine, bromine and iodine. A haloalkyl group typically has the formula CₙH₍₂ₙ₊₁₋ₘ₎Xₘ wherein each X is independently selected from the group consisting of F, Cl, Br and I. In groups of this type n is typically from 1 to 20, from 1 to 10 or from 1 to 6. m is typically 1 to 10, 1 to 6 or 1 to 3. Examples of haloalkyl include fluoromethyl, difluoromethyl and trifluoromethyl.

The term alcohol as used herein refers to a hydrocarbon having one or more hydroxyl (—OH) moieties with at least one carbon atom, 1 to 20 carbon atoms, 2 to 20 carbon atoms, 2 to 10 carbon atoms or any number of carbon atoms falling within any of these ranges. Exemplary alcohols may include, but are not limited to, ethanol, propanol, butanol, pentanol or other substituted alcohols.

The term “carbonyl” as used herein refers to a hydrocarbon that has a R₁—C(═O)—R₂ group, wherein both R₁ and R₂ may be independently a hydrogen or any of the optional substituent groups as defined above. Such carbonyls may include aldehydes or ketones.

The ethanol (EtOH) conversion used herein is derived as follows:

$$\frac{{n}_{EtOH i n} - {n}_{EtOH out}}{{n}_{EtOH i n}} \times 100 \%$$

The butadiene (BD) selectivity as used herein is derived as follows:

$$\frac{{n}_{BD}}{{n}_{EtOH i n} - {n}_{EtOH out}} \times 2 \times 100 \%$$

The weight hourly space velocity (WHSV) as used herein is derived as follows:

$$\frac{{mass}_{EtOH per hour}}{{mass}_{reactor 1 catalyst} + {mass}_{reactor 2 catalyst}} ,$$

wherein reactor 1 refers to the first reaction zone and reactor 2 refers to the second reaction zone.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF EMBODIMENTS

Exemplary, non-limiting embodiments of a method for producing an optionally substituted olefin, will now be disclosed.

In one embodiment, there is provided a method for producing an optionally substituted olefin, comprising the steps of: dehydrogenating an optionally substituted alcohol in a first reaction zone comprising a first catalyst supported on a porous silica-based particle to form an optionally substituted carbonyl at a first set of reaction conditions; converting the optionally substituted alcohol and the optionally substituted carbonyl from the first reaction zone in a second reaction zone at a second set of reaction conditions that is different to the first set of reaction conditions and is selected to form the optionally substituted olefin, wherein the second reaction zone comprises a second catalyst supported on a porous silica-based particle.

An exemplary mechanism for the alcohol to olefin reaction, represented by an ethanol to butadiene reaction, is shown in FIG. 1. The mechanism may involve the conversion of ethanol to acetaldehyde via a dehydrogenation path (step 1 in FIG. 1), followed by the aldol condensation of acetaldehyde and then dehydration to crotonaldehyde (step 2 in FIG. 1), which undergoes a Meerwein-Ponndorf-Verley reduction with ethanol to crotyl alcohol and then dehydration to yield butadiene (step 3 of FIG. 1). Advantageously, the ratio of acetaldehyde (or crotonaldehyde) to ethanol as reaction feed to the third step of FIG. 1 may improve the overall butadiene yield. Furthermore, to reduce the carbon footprint of producing, purifying and/or storing acetaldehyde, followed by mixing it with ethanol in separate processes, a fixed, optimized ratio of acetaldehyde to ethanol may be generated in situ in a single process as disclosed herein for use as reaction feed to the third step of FIG. 1, thereby providing a more cost-effective and green reaction.

Accordingly, in the disclosed method, the reaction conditions of the dehydrogenating step may be controlled to achieve an optimum alcohol to carbonyl molar ratio. Advantageously, the selectivity and overall yield of the final olefin product may be optimized.

The first set of reaction conditions in the first reaction zone may comprise controlling the alcohol/carbonyl molar ratio provided to the second reaction zone. The ratio between the optionally substituted alcohol and the optionally substituted carbonyl provided to the second reaction zone may be controlled to achieve optimum selectivity of the produced optionally substituted olefin. The ratio between the optionally substituted alcohol and the optionally substituted carbonyl may be varied by fixing the temperature of the second reaction while varying the temperature of the first reaction. The ratio between the optionally substituted alcohol and the optionally substituted carbonyl may be controlled by fixing the temperature in the second reaction zone while controlling the temperature in the first reaction zone. The optimum ratio of alcohol/carbonyl may be obtained by analyzing the output stream of the first reaction at that particular temperature that gives the highest olefin selectivity.

Control of the alcohol/carbonyl ratio afforded by the robust catalyst for the dehydrogenation reaction and/or control of the dehydrogenation reaction temperature condition provide for an improved process. The disclosed catalysts may be substantially dispersed on the porous silica-based support which may have relatively large nano-sized pores. Advantageously, the feed stock to the second reaction zone and the preparation of the catalyst and support material are optimized to enhance alcohol conversion and olefin selectivity.

The dehydrogenating step may be conducted at conditions suitable to increase the selectivity towards the carbonyl, while decreasing the selectivity towards other by-products.

The first set of reaction conditions may comprise conducting the dehydrogenating step at a temperature of between 100° C. to 500° C., or 100° C. to 400° C., or 200° C. to 320° C., or at any temperature within these ranges. The dehydrogenating step may be undertaken at about 220° C., or about 225° C., or about 230° C., or about 235° C., or about 240° C. At these temperatures, the selectivity of the resultant optionally substituted olefin may be maximized without compromising the conversion percentage of the optionally substituted alcohol feedstock.

The alcohol feedstock may be provided to the first reaction zone in a form suitable to optimize its contact with the first supported catalyst. The alcohol feedstock may be provided in the vapor form.

The alcohol feedstock may be provided to the first reaction zone in an aqueous solution. The alcohol feedstock may be provided to the first reaction zone as a dry feedstock. The alcohol may be substantially pure alcohol, whereby the water content in the alcohol feedstock is negligible. The water content in the alcohol feed may be less than 0.05 v/v %, or less than 0.01 v/v %, or less than 0.005 v/v %. The water content in the alcohol feed may be less than 20 v/v %, or less than 15 v/v %, or less than 10 v/v %. The alcohol feedstock may be aqueous alcohol having a water content of 10 vol %.

The first supported catalyst may comprise a single metal catalyst, binary metal catalyst, ternary metal catalyst or any of their equivalent metal oxide catalyst. The metal or metal oxide for this first supported catalyst may be selected from the group consisting of silver, gold, copper, zinc, aluminum, magnesium, zirconium, tantalum, titanium, vanadium and their combinations thereof. This first supported catalyst may be a copper-MCF supported catalyst.

The first reaction zone may be provided as a packed bed or fixed bed reactor comprising the first supported catalyst. The first reaction zone may be provided as a fluidized bed comprising the first supported catalyst.

The silica-based particle may be a siliceous foam. The siliceous foam may be macroporous, microporous or mesoporous. Particularly, the siliceous foam may be mesoporous and capable of having a mesocellular network. This mesocellular porous siliceous foam (MCF) may have any regular or irregular shape, wherein the regular shaped mesoporous siliceous particles may be spherical, cylindrical, oblong or ellipse. This MCF may have a pore size and surface area as disclosed herein, e.g. a pore size in the range of 2 and 50 nm and a surface area of at least 350 m²/g. By using this MCF as a support for the first and second catalysts, the catalytic activity of these catalysts may be enhanced due to the large pore size of the MCF which aids to increase contact between the catalysts and the reactants.

The optionally substituted alcohol may be any alcohol capable of being dehydrogenated to form an optionally substituted carbonyl. Exemplary alcohols may include, but are not limited to, ethanol, propanol, butanol, pentanol or any other substituted alcohol. The optionally substituted alcohol may have at least 2 carbon atoms.

The optionally substituted olefin may be an optionally substituted diene. The optionally substituted olefin or diene may have at least 4 carbon atoms. The optionally substituted olefin or diene may have 4 to 20 carbon atoms. Exemplary dienes may include, but are not limited to, butadiene, pentadiene, hexadiene, heptadiene, octadiene and nonadiene, or other substituted dienes.

The optionally substituted carbonyl may be any ketone or aldehyde, such as but not limited to, acetaldehyde or crotonaldehyde. This optionally substituted carbonyl may have the same number of carbon atoms as the optionally substituted alcohol from which it is derived.

The optionally substituted alcohol may be ethanol, the optionally substituted olefin may be butadiene and the optionally substituted carbonyl may be acetaldehyde.

The second set of reaction conditions is different to the first set of reaction conditions and is selected to form the optionally substituted olefin. The second set of reaction conditions may comprise conducting the conversion step at a temperature of between 250° C. to 550° C. or any temperatures falling within this range. Particularly, the conversion may occur at about 375° C., or about 400° C. Advantageously, these temperatures may maximize the selectivity of the resultant optionally substituted olefin without compromising the conversion percentage of the optionally substituted alcohol feedstock.

The converting step may further comprise a step of coupling the optionally substituted alcohol with the optionally substituted carbonyl, and a step of dehydrating the coupled optionally substituted alcohol and optionally substituted carbonyl to form the optionally substituted olefin.

The first catalyst and the second catalyst may be different metal catalysts composed of different metals.

The second supported catalyst may be a single metal catalyst, binary metal catalyst, ternary metal catalyst or any of their equivalent metal oxide catalyst. The metal or metal oxide for the second supported catalyst may be selected from the group consisting of silver, gold, copper, zinc, aluminum, magnesium, zirconium, tantalum, titanium, vanadium and their combinations thereof. Particularly, the second supported catalyst may be a zirconium-MCF supported catalyst.

The second reaction zone may be provided as a packed bed or fixed bed reactor comprising the second supported catalyst. The second reaction zone may be provided as a fluidized bed comprising the second supported catalyst.

The first and second supported catalysts may be capable of regeneration. For example, the disclosed supported catalysts may be regenerated by calcination.

The disclosed method advantageously comprises the use of two reaction zones that are fluidly connected. The provision of two separate but fluidly connected reaction zones advantageously enables the optimization of the reaction conditions for each reaction. The use of a dual reactor system in the present method allows the ratio of the optionally substituted alcohol and the optionally substituted carbonyl to be optimally achieved for feeding to the second reactor, thereby achieving optimum selectivity of the produced optionally substituted olefin. The utilization of the disclosed dual reactor system advantageously reduces the need for purifying, storing or mixing the carbonyl with the optionally substituted alcohol which is the case if the reactors for the dehydrogenating step and the converting step are separated. Accordingly, the dual reactor system and method may provide a more cost-effective and environmentally friendly means for producing an optionally substituted olefin from an optionally substituted alcohol, such as in the production of butadiene from ethanol.

Exemplary, non-limiting embodiments of a system for producing an optionally substituted olefin, will now be disclosed.

In an embodiment, there is provided a system for producing an optionally substituted olefin, the system comprising: a first reaction zone having a first catalyst for dehydrogenating an optionally substituted alcohol to form an optionally substituted carbonyl; and a second reaction zone having a second catalyst for converting the optionally substituted alcohol and the optionally substituted carbonyl from the first reaction zone to form the optionally substituted olefin; wherein the first and second catalysts are supported on a porous silica-based particle.

Advantageously, the porous silica-based particles disclosed above are capable of enhancing the conversion yield of an optionally substituted alcohol to an optionally substituted olefin without compromising the selectivity of the intermediate optionally substituted carbonyl. Such an advantage may be attained through the porous silica-based particles which provide an improved catalytic activity due to its enhanced pore size or porosity.

On the other hand, typical silica gels are not able to improve the catalytic activity and conversion yield. These silica gels can be distinguished from the porous silica-based particles as disclosed above. Although the particles and the particles used for forming silica gels are porous with broad pore size distribution, the porosity or pore size within the silica gels are not capable of providing the above advantages. This is because typical metal catalysts supported by silica gels are prepared by impregnation methods which result in the formation of the oxides of the metal catalysts having relatively larger particle sizes. These larger sized metal oxide particles potentially reduce the pore size and the porosity of the silica gel. Hence, metal catalysts supported on silica gels suffer from reduced catalytic activity as a result of the reduction of pore size or porosity of such silica gels. Accordingly, the metal catalyst itself is also compositionally affected in the sense that the amount of the original metal catalyst is reduced due to the oxide formed from the impregnation method, thereby resulting in lower conversion yield. Reported catalysts comprising binary or ternary metal-metal oxide components supported on silica gel suffered from low ethanol conversion averaging about 33% and low weight hourly space (WHSV) velocity of 0.3 hr⁻¹ even though the highest butadiene yield attained per mole of starting ethanol was only at 81%. Moreover, when the WHSV is increased to 15 hr⁻¹, both the ethanol conversion and butadiene selectivity decreased to 12% and 67%, respectively. Hence, silica gel-supported catalysts are not efficient or effective based on these reported results.

Meanwhile, conventional silicate particles used as supports are not sufficiently porous or capable of having a mesocellular network within the particles, which is in contrast to the presently disclosed silica-based particle support. Hence, conventional discrete silicate particles, which are structurally distinct from the mesoporous silica-based particles as disclosed herein, remain deficient in enhancing catalytic activity and conversion yield. These discrete silicate particles are typically formed by mixing a metal, silicon and oxygen which then undergo condensation in water to arrive at the abovementioned discrete silicate particles. This means that not all the metals may be successfully immobilized on the silicate particle surface. Hence, such discrete silicate particles suffer from reduced catalytic activity due to less metal catalysts being immobilized thereon.

In contrast, the present silica-based particles are formed before the metal catalysts are immobilized thereon. Therefore, more metal catalysts may be successfully immobilized on the silica-based particles. Along with the enhanced pore size and porosity of the present silica-based particles, more metal catalysts on such a substrate may be exposed for maximum interaction with reactants, which leads to increased catalytic activity for higher conversion yield.

Notably, both silica gel and discrete silicate particles have different particle morphologies compared to the present silica-based particle.

The present porous silica-based particle may be a siliceous particle. The silica-based particle or the siliceous particle may be macroporous or mesoporous.

A macroporous particle may have a typical pore size that is greater than the pore size of a mesoporous particle, while a microporous particle may have a typical pore size smaller than that of a mesoporous particle.

In some embodiments, the disclosed particle may be mesoporous. The disclosed particle may be mesoporous silica. Mesoporous silica has a wide variety of applications and is extensively used in catalysis. The disclosed particle may be highly porous, comprising an inter-connected network of uniform pores within each particle, and is structurally different compared to silica gel and discrete silicate particles. For example, the disclosed particle may have a sponge-like pore structure that is absent in discrete silicate particles or particles used for forming silica gel. The discrete silicate particles or particles used for forming the silica gel tend to be rigid in the sense that it may not be able to change its shape without the particle breaking apart into smaller pieces of particles. On the other hand, the present silica-based particle having a sponge-like pore structure may be deformable in the sense that the silica-based particle may be able to change its shape without breaking apart into smaller particles. In other embodiments, the present silica-based particle may not be deformable.

The silica-based particle or the mesoporous siliceous particle may have a mesocellular network within the particle. This mesoporous particle may be referred to as a porous siliceous foam or mesocellular siliceous foam (MCF). Accordingly, the porous silica-based particle may be a porous silica-based foam. Mesostructured cellular foams are composed of uniformly sized, large spherical cells that are interconnected by uniform, smaller-sized pores called windows, to create a continuous 3-D pore system. The interconnected nature of the large uniform pores makes these new mesostructured silicas promising candidates for use as catalyst supports due to the high surface area available to support the catalyst. Additionally, the interconnected network of pores may permit the reaction fluids to flow through the pores of the support and contact the catalysts dispersed on the inner surfaces of the pores. The mesocellular porous siliceous foam may be made up of particles comprising any regular or irregular shape.

The disclosed silica-based particles may be of regular or irregular shape. Regular shaped particles may be spherical, cylindrical, oblong or ellipse. These particles may be micro-particles. These particles may have a size of at least 1 um. These particles may also have a size between 1 um to 20 um, or between 1 um to 15 um, or between 1 um to 10 um, or between 5 um to 20 um, or between 5 um to 15 um. The term “particle size” means an average axial length, e.g. diameter, of the particle.

The pore size of the mesoporous siliceous foam may be in the range of 1 nm to 100 nm, or more particularly 5 nm to 50 nm, or 10 nm to 40 nm, or 25 nm, or 29 nm, or 30 nm, or 35 nm. The pore size of the window pores may be in the range of 0.1 nm to 100 nm, or 2 nm to 100 nm, or 10 nm to 50 nm, or 10 nm to 20 nm, or 14 nm, or 15 nm, or 16 nm. Pore size may be positively related to olefin selectivity and productivity. A larger pore size may advantageously be less sensitive to coking. Therefore, catalysts supported on mesoporous silica or mesocellular siliceous form may possess better longevity than catalysts supported on conventional silica.

The surface area of the disclosed silica-based particle may be at least 300 m²/g, or at least 350 m²/g, or more than 400 m²/g, or more than 500 m²/g.

Advantageously, the silica-based particles as defined above is capable of acting as a mesoporous support for the first and second catalysts, in which the activity of both catalysts may be enhanced due to its improved pore size and porosity.

The disclosed silica-based particle may comprise a silica content of from 90 to 100 weight percent. The disclosed silica-based particle may comprise substantially of silica.

The optionally substituted olefin may be an optionally substituted diene. The optionally substituted olefin or optionally substituted diene may have at least 4 carbon atoms. The optionally substituted olefin or optionally substituted diene may have 4 to 20 carbon atoms or any number of carbon atoms falling within this range. Exemplary dienes may include, but are not limited to, butadiene, pentadiene, hexadiene, heptadiene, octadiene and nonadiene, or other substituted dienes.

The optionally substituted alcohol may be any alcohol capable of being dehydrogenated to form an optionally substituted carbonyl. Exemplary alcohols may include, but are not limited to, ethanol, propanol, butanol, pentanol or any other substituted alcohol. The optionally substituted alcohol may have at least 2 carbon atoms. The optionally substituted alcohol may have a number of carbon atoms falling within the range of 2 to 20.

The optionally substituted carbonyl may be any carbonyl compound capable of being converted to an optionally substituted olefin, e.g. diene. This optionally substituted carbonyl may be an aldehyde or a ketone. The optionally substituted carbonyl intermediate may comprise one or more types of carbonyl intermediates, depending upon the reactants used, the reaction and its kinetics. The optionally substituted carbonyl may have the same number of carbon atoms as the optionally substituted alcohol from which it is derived, or may have different number of carbon atoms as the optionally substituted alcohol from which it is derived depending on the reaction pathway. The one or more types of carbonyl intermediates may have the same or different number of carbon atoms as the optionally substituted alcohol from which it is derived. Where there is more than one type of carbonyl intermediates, the carbonyl intermediates may have the same or different number of carbon atoms as the other intermediates.

For example, where the alcohol is ethanol, the optionally substituted carbonyl may be acetaldehyde and/or crotonaldehyde. Crotonaldehyde may be formed as a by-product in the first reaction zone during dehydrogenation. The crotonaldehyde may be formed due to the condensation of acetaldehyde. In the embodiment where a copper catalyst is used as the first catalyst, it is expected that crotonaldehyde may be produced as copper is slightly basic. However, this crotonaldehyde formed may be in minute quantities as the dehydrogenation of the optionally substituted alcohol tends to be selective towards the formation of acetaldehyde.

The conversion of ethanol to butadiene typically uses catalysts such as silica- and alumina-based single, binary, or ternary metal oxides, e.g. copper, zinc, zirconium, tantalum and magnesium oxides. In an example, a silver oxide/magnesia/silica ternary catalyst may be used with pure ethanol to afford a 45.4% butadiene yield at an ethanol conversion of 91.8%. In another example, hydrogen peroxide may be used to initiate a zinc oxide/γ-Al₂O₃ catalyst over an ethanol/hydrogen peroxide solution to afford a butadiene yield of 24.5% and a selectivity of 55%. In yet another example, a tantalum (Ta) oxide catalyst supported on ordered mesoporous silica may be used to afford an ethanol conversion of 47% and a butadiene selectivity of 79% after 10 hours time-on-stream. In another example, a reduced copper on alumina catalyst may be used for the dehydrogenating step and a tantalum oxide on silica catalyst may be used for the conversion step to produce butadiene at a yield of 23% and a butadiene productivity of 43 g of butadiene per litre of catalyst per hour over 20 hours.

In contrast, the disclosed supported catalysts may advantageously result in a higher butadiene yield than prior art examples. For example, the disclosed supported catalysts may afford the disclosed method with an ethanol conversion of between 60% and 95% and an acetaldehyde selectivity of from 85% to more than 95% or more than 97%. The overall butadiene yield may be more than 60%, more than 70%, or more than 73%. The butadiene yield may be analysed by a suitable analysis method, such as by gas chromatography. The analysis method may utilize a suitable detector, such as a thermal conductivity detector or a flame ionization detector. In an embodiment, the detector is a flame ionization detector which may be able to detect butadiene among other molecules with four carbons. In an example, using gas chromatography equipped with flame ionization detector (GC-FID), the overall butadiene yield is in excess of 73% over 15 hours at an ethanol WHSV of 1.5 hr⁻¹ using nitrogen as carrier gas with substantially pure ethanol. In another example, using GC-FID, the overall butadiene yield is in excess of 71% over 15 hours at an ethanol WHSV of 1.5 hr⁻¹ using nitrogen as carrier gas with aqueous ethanol at 10 vol % water.

The first catalyst may be a single metal catalyst, binary metal catalyst, ternary metal catalyst or any of their equivalent metal oxide catalyst, i.e. single metal oxide catalyst, binary metal oxide catalyst or ternary metal oxide catalyst. The metal or metal oxide of the first catalyst may be selected from the group consisting of silver, gold, copper, cobalt, zinc, aluminum, magnesium, manganese, zirconium, tantalum, titanium, vanadium and their combinations thereof. The first catalyst may be copper supported on the porous silica-based particle. The first catalyst may be a binary catalyst comprising two metals or metal oxides listed above. The first catalyst may be a ternary catalyst comprising three metals or metal oxides listed above.

Particularly, the first catalyst may be a copper-MCF supported catalyst. Advantageously, copper serves as a relatively cheaper catalyst and is more abundant when compared to other metals such as gold, silver or vanadium.

The second catalyst may be a single metal catalyst, binary metal catalyst, ternary metal catalyst or any of their equivalent metal oxides catalyst, i.e. single metal oxide catalyst, binary metal oxide catalyst or ternary metal oxide catalyst. The metal or metal oxide of the second catalyst may be selected from the group consisting of silver, gold, copper, zinc, aluminum, magnesium, zirconium, tantalum, titanium, vanadium, cerium and their combinations thereof. The second catalyst may be zirconium supported on the porous silica-based particle. The second catalyst may be a binary catalyst comprising two metals or metal oxides listed above. The second catalyst may be a ternary catalyst comprising three metals or metal oxides listed above. In an embodiment, the second catalyst may be a binary catalyst having the general formula M₁/M₂, wherein M₁ and M₂ are independently selected from the group listed above. In an example, M₁ is zirconium and M₂ is selected from the group consisting of cerium (Ce), copper (Cu), magnesium (Mg) and zinc (Zn).

Particularly, the second catalyst may be a zirconium-MCF supported catalyst. For the same reasons as described above in relation to the copper-MCF supported catalyst, zirconium may be selected as the second catalyst as it is relatively more affordable. Further, zirconium provides better catalytic performance and the synthesis procedure for zirconium-MCF catalyst is more straightforward for scaling up.

The disclosed supported catalysts may comprise a metal content of about 1 to 15 weight percent, or about 1 to 10 weight percent, or about 1 to 8 weight percent, and a metal oxide content of about 1 to 20 weight percent, or about 1 to 15 weight percent, or about 1 to 10 weight percent. In embodiments, the disclosed catalysts comprise substantially of metal oxide. In other embodiments, the disclosed catalysts comprise substantially of metal. The catalyst may be of a particle size and/or surface area optimized to permit contact of the catalyst surface and the reactants. The particle size of the catalyst may be optimized having regard to the size of the pores of the silica-based particle support. The catalyst particles may be of a size that does not hinder reactants from contacting catalyst dispersed on the inner surfaces of the support. The metal and/or metal oxide of the first catalyst, if present, or the metal and/or metal oxide of the second catalyst, if present, may independently be of a size that is less than 2 orders of magnitude, or less than 3 orders of magnitude, or less than 4 orders of magnitude, smaller than the size of the silica-based particle (and therefore smaller than the pores of the silica-based particle) so as not to hinder reactants from contacting catalyst immobilized on the inner surfaces of the porous silica-based particle support. The disclosed metal oxide catalysts, if present, may advantageously be of a smaller particle size as compared to metal oxide catalysts produced by an impregnation method. In an example, the size of the metal oxide catalyst may be 5 nm or smaller when the silica-based particle has a size of 1 um. In other examples, the size of the metal and/or metal oxide catalyst may be 5 nm or smaller, or 4.5 nm or smaller, or 4 nm or smaller. Advantageously, the size and composition of the catalyst particles may be controlled to optimize the conversion yield to the olefin.

The disclosed supported catalyst may be a heterogeneous catalyst. The disclosed supported catalyst may comprise a silica content of 90 weight percent or more, or 92 weight percent or more, or 95 weight percent or more. The disclosed supported catalyst may comprise a metal and/or metal oxide content of 10 weight percent or less, or 9 weight percent or less, or 8 weight percent or less, or 7 weight percent or less, or 5 weight percent or less. The disclosed supported catalyst may comprise a silica content of from 90 to 100 weight percent and a metal and/or metal oxide content of from 0 to 10 weight percent.

The first supported catalyst may comprise more than 3 mol %, more than 3.5 mol %, or more than 4 mol %, of metal and/or metal oxide content. In an example, where the first catalyst is copper and the porous silica-based particle is MCF, the copper loading of the Cu/MCF catalyst may be 4 mol % or 4.1 mol % when analysed with inductively coupled plasma mass spectrometry (ICP-MS).

The second supported catalyst may comprise more than 1 mol %, more than 1.5 mol %, more than 2 mol %, more than 2.5 mol %, or more than 3 mol %, of metal and/or metal oxide content. In an example, where the second catalyst is zirconium and the porous silica-based particle is MCF, the zirconium loading of the Zr/MCF catalyst may be 2 mol % when analysed with ICP-MS.

The disclosed supported catalyst may be synthesized by mixing a precursor of the catalyst with a suspension of the disclosed porous silica-based particle support, which may be synthesized according to literature procedure. The catalyst precursor may be soluble in an aqueous solution. The catalyst precursor may be metal ions. The catalyst precursor may be a metal salt or a mixture of metal salts, wherein the metal is as disclosed herein. The solution or suspension of the silica-based particle may be an aqueous one.

The synthesis method of the supported catalyst may be conducted in a solution at a pH of more than 7, i.e. in an alkaline environment. The alkaline environment confers a negative charge on the surface of the silica-based particle. The alkaline environment may have a pH of more than 7, or about 7.5, or about 8, or about 8.5, or about 9, or about 9.5, or about 10.

Basic compounds such as ammonia or urea may be added into the solution to increase the pH. The negatively-charged silica-based particle may attract the metal precursor by chemisorption to thereby achieve improved dispersion of the catalyst on the silica-based particle support. The mixture may be purified, e.g. by filtering and drying, and then be heated to form the disclosed supported catalyst. The heating may be conducted in the presence of air. The heating may oxidize the catalyst on the support. The heating may produce metal oxide catalyst particles on the support. The heating may not decompose the catalyst particles. The oxidized catalyst may at least partially be reduced so that the catalyst immobilized on the support may comprise both the oxidized catalyst and original pure catalyst. The reduction may be typically conducted in the presence of hydrogen at elevated temperatures for a few hours.

The disclosed supported catalyst may comprise a support/oxidized catalyst interface and an oxidized catalyst/pure catalyst interface. The part of the catalyst exposed to the reaction environment may be the reduced or pure catalyst. For example, the disclosed supported catalyst may comprise a metal oxide/silica interface where the silica is bonded to the metal oxide via oxygen bridging, and wherein the outer part of the metal oxide may be reduced to pure metal by a reduction process.

As the disclosed silica-based particle support may be highly porous, a substantial percentage of the support surface, such as more than 90% of the surface or more than 95% of the surface, may be within the particle. That is, a substantial percentage of the surface area of the silica-based particle support may comprise the surfaces of the interconnected network of pores within the particle.

Advantageously, the catalyst may be grown or immobilized or supported on surfaces that are substantially within the particle support. Further advantageously, a substantial amount of catalyst supported on the surfaces of the pores within the particle support may be of a relatively small size, such as 5 nm or smaller. The pore size of the particle support may advantageously not be reduced as much compared to prior art supported catalysts, thereby improving the catalytic activity. Yet further advantageously, part of the catalyst exposed to the reactants may be the original pure catalyst. Hence, deactivation of the catalyst may be reduced as compared to conventional catalysts due to a higher exposure of original catalyst to the reaction environment. In any event, even if part of the catalyst exposed to the reactants may be the oxidized catalyst, the oxidized catalyst may not be fully decomposed, thereby retaining its catalytic activity.

The amount of basic compound added may be in mole excess to the amount of catalyst precursor. The molar ratio of the catalyst precursor to the basic compound may be 1:5. Where urea is used, the mole ratio of the catalyst precursor to the basic compound may be about 1:10. The urea may be hydrolysed and thermally decomposed during heating. The thermal decomposition of urea in the aqueous solution produces ammonia which increases the pH of the solution. The alkaline environment aids in the gradual precipitation of the catalyst precursor onto the surfaces of the porous silica-based particle. For example, a zirconium precursor, e.g. ZrOCl₂.8H₂O, may be gradually precipitated to zirconium hydroxide onto the surfaces of the pores of the silica-based particle.

The disclosed synthesis method may be conducted for a duration of three hours or more.

Advantageously, the disclosed synthesis method controls the precipitation process of the catalyst to result in smaller catalyst particles. Advantageously, the disclosed synthesis method may be conducted in an alkaline environment to result in improved dispersion of the catalyst particles on the support surface.

Advantageously, the disclosed catalyst is formed on the surfaces of the silica-based particle. Accordingly, the disclosed method may advantageously result in the catalyst being much more dispersed on the porous silica-based particle support as compared to prior art catalysts. The disclosed supported catalyst comprising a catalyst supported on a porous silica-based particle may have improved catalytic activity and stability as compared to prior art catalysts. The disclosed method may advantageously result in a supported catalyst that retains the catalytic activity of the metal. The disclosed method may advantageously result in a lower amount of metal oxide formed, thereby producing an improved supported catalyst system. The disclosed method may advantageously immobilize more metal catalysts on the porous support simply by chemisorption and without complicated reaction pathways or the use of additives.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 is an exemplary reaction scheme for the conversion of ethanol to butadiene.

FIG. 2 shows the nitrogen isotherms of the prepared catalysts and a blank MCF support referred to in Example 1.

FIG. 3 shows the X-ray diffraction (XRD) spectrum of the prepared catalysts and a blank MCF support referred to in Example 1.

FIG. 4 is a schematic illustration of the experimental setup used in Examples 2-4.

FIG. 5 is a graph of the ethanol conversion and the BD selectivity against the temperature of the catalysts in Example 2.

FIG. 6 is a graph of the ethanol conversion and the acetaldehyde selectivity against reaction time of the Cu/MCF catalyst in (a) pure ethanol and (b) in aqueous ethanol, in Example 3.

FIG. 7 is a graph of the ethanol conversion and the butadiene selectivity of entry 1 of Example 4 against reaction time of the dual reaction system in (a) pure ethanol and (b) in aqueous ethanol.

FIG. 8 is a graph of the ethanol conversion and the BD selectivity against reaction time when regenerated catalyst was used in Example 2.

FIG. 9 is a schematic illustration of the experimental setup used in Example 5.

FIG. 10 is a schematic illustration of the experimental setup used in Examples 6 and 8.

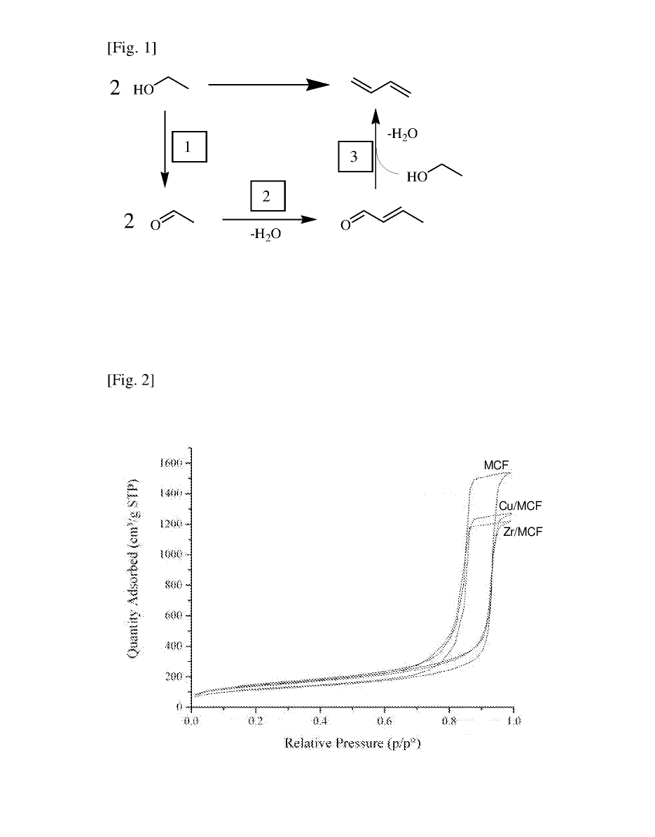

### EXAMPLES

Non-limiting examples of the invention will be further described in greater detail by reference to specific Examples, which should not be construed as in any way limiting the scope of the invention.

### Example 1

### Preparation of the Cu Catalyst on MCF (Cu/MCF)

30 mL of deionized (DI) water was added to 1 g of MCF synthesized according to a known method (Y. Han, S. S. Lee and J. Y. Ying, Chemistry of Materials, 2007, 19, 2292-2298). An appropriate amount of soluble copper precursor (e.g CuNO₃ or CuCl₂) was added into the MCF/water mixture and was rapidly stirred. A solution of aqueous ammonia (4 M) was added dropwise until the pH reached ˜9. At pH 9, the silica surface was negatively-charged and attracted the positively-charged [Cu(NH₃)₄]²⁺ species in the solution.

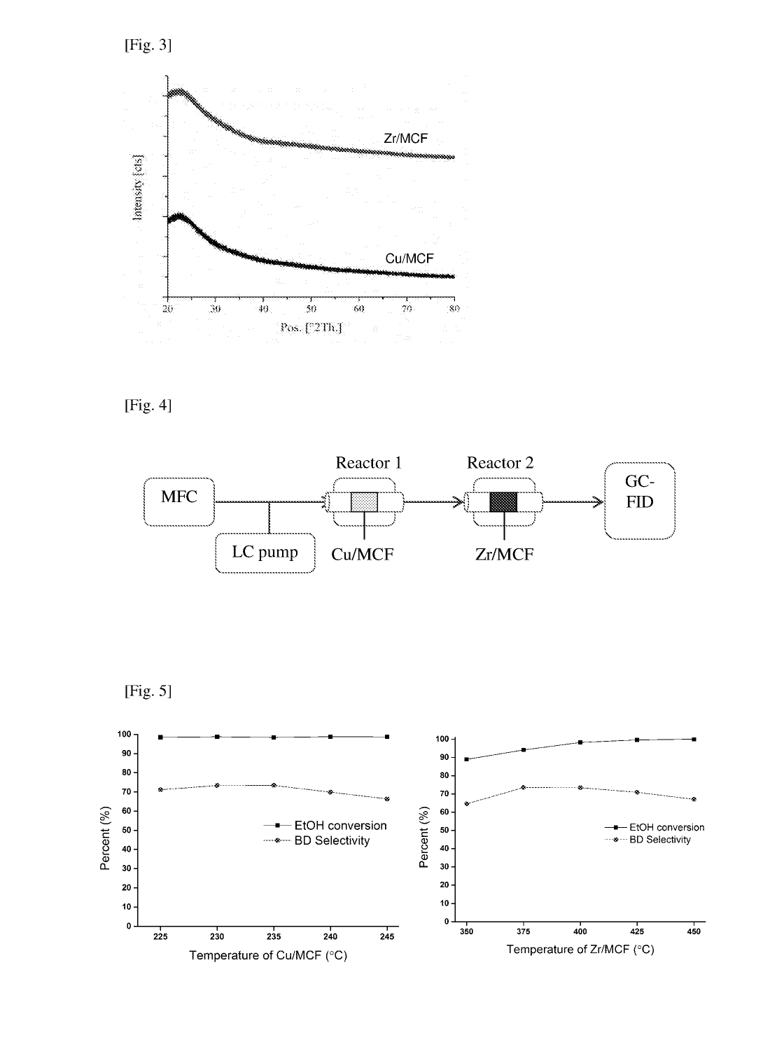

The mixture was stirred for 10 min, filtered, washed several times with water, and then dried under vacuum for 12 hours. The resulting powders were heated at 500° C. for 3 hours to obtain the final green coloured Cu/MCF catalyst. Formation of highly-dispersed copper on an MCF support was achieved. The Cu/MCF catalyst did not show any visible peaks in its XRD spectrum (FIG. 3) confirming the highly dispersed copper elements on the surface of MCF.

However, the Cu-im-MCF catalyst prepared by the prior art impregnation method showed a typical XRD pattern of CuO (not shown), which indicates that relatively big CuO particles were formed on the MCF surface.

### Preparation of the Zr Catalyst on MCF (Zr/MCF)

20 mL of DI water was added to 1 g of MCF. The urea hydrolysis method was used for the preparation of the Zr/MCF catalyst. An appropriate amount of zirconium precursor (ZrOCl₂.8H₂O or ZrONO₃.xH₂O) and urea in a mole ratio of 1 to 10 was added to the MCF/water mixture and was rapidly stirred. The resultant mixture was heated up to 90° C. and stirred for 6 hours. After cooling, the mixture was filtered, washed several times with water, and then dried under vacuum for 12 hours. The resulting powders were heated at 500° C. for 3 hours to obtain the final colourless Zr/MCF catalyst.

### Characterization

The prepared supported catalysts and a blank MCF support were analyzed.

Nitrogen isotherms were measured at −196° C. and P/Po between 0.01 and 0.995 on a Micromeritics ASAP 2020 (Georgia, USA) after degassing samples of the catalysts at 200° C. under vacuum overnight. The measured nitrogen isotherms are shown in FIG. 2.

The surface areas of all samples were calculated using the Brunauer-Emmett-Teller (BET) equation. The Barret-Joyner-Halenda (BJH) method was used to calculate the pore size distribution of the samples using data obtained from the nitrogen isotherm. The results are shown below in Table 1.

TABLE 1

| | BET Surface | BJH | BJH |
|---|---|---|---|
| Material | area (m²/g) | Adsorption (nm) | Desorption (nm) |
| MCF | 512 | 31.0 | 13.3 |
| Cu/MCF | 402 | 30.0 | 13.3 |
| Zr/MCF | 477 | 28.5 | 12.8 |

Powder X-ray diffraction (XRD) patterns were obtained using a Philips X'pert Pro diffractometer equipped with a CuKα radiation source (1.506 \acute{Å}), operating at a 2θ range of 20° to 80°. Inductively coupled plasma mass spectrometry (ICP-MS) analyses were performed using Perkin-Elmer Elan DRC II (Massachusetts, USA) on HF/HNO₃-digested samples and appropriate standard solutions. The XRD data is shown in FIG. 3.

### Example 2

### Catalytic Reaction by the Dual Reactor System

Ethanol, acetaldehyde, diethyl ether, crotonaldehyde, crotyl alcohol were calibrated by manual injection (average of five injections) of known amounts into a gas chromatography (GC) machine equipped a thermal conductivity detector (FID). Ethylene and BD were calibrated using a certified gas blend of 2 mol % each in nitrogen balance. Both gases were injected into the GC using a 250 uL gas syringe.

A liquid chromatography (LC) pump was used to control amount of ethanol in the system as disclosed herein.

A mass flow controller (MFC) was used to control the rate of nitrogen flow, which delivered vaporized ethanol through a fixed bed reactor packed with 20 mg of Cu/MCF (the first reaction zone). The resultant stream of gas was then delivered to another fixed bed reactor packed with 60 mg of Zr/MCF (the second reaction zone). A schematic illustration of the setup is shown in FIG. 4.

Initial ethanol amount in the flow was determined via sampling using a gas valve system at 150° C. in both reactors. The reaction was carried out at ambient pressure and monitored at 1 hr intervals. The outlet products were analyzed periodically using GC-FID with a 30 m-long PoraPlot Q column via the gas valve system. Output gas was bubbled into CDCl₃ for ¹H NMR spectroscopy and analysed for qualification purposes.

The reaction temperature of both the supported catalysts in reactors 1 and 2 were optimized by plotting the ethanol conversion and the BD selectivity against the temperature of the catalysts, which represents the temperature of the reactor. The first data point was obtained at 100 mins into the reaction. The Cu loading of the Cu/MCF catalyst was 4.1 mol % and the Zr loading of the Zr/MCF catalyst was 2.0 mol %. The results are shown in FIG. 5.

From the obtained results, the optimum temperature for Cu/MCF and Zr/MCF is 235° C. and 400° C., respectively.

The catalyst was regenerated by calcination under air at 500° C. for 3 hours.

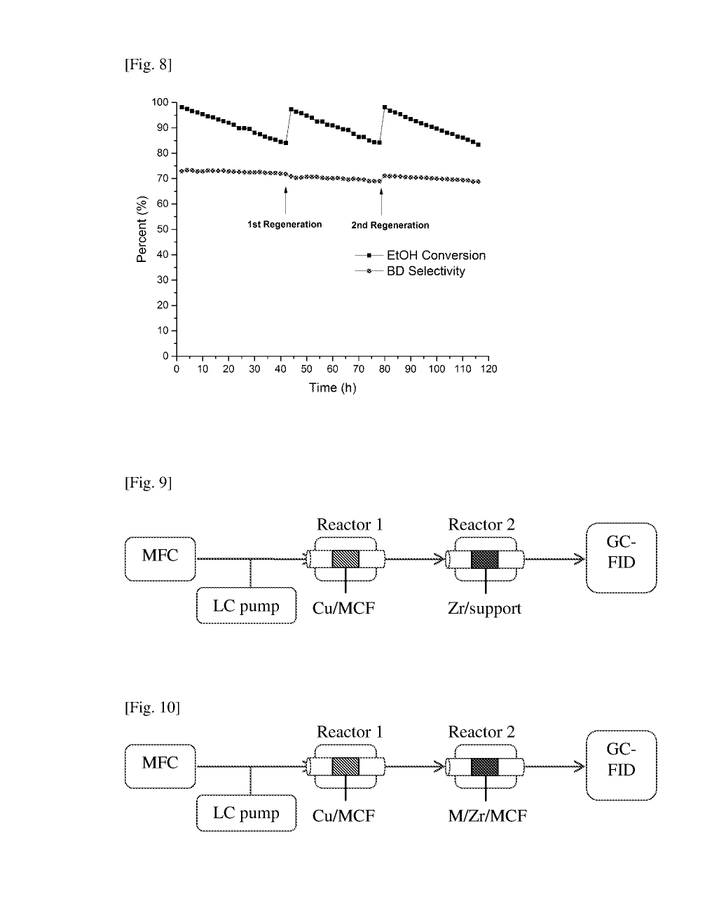

The experiment was repeated with regenerated catalysts and the ethanol conversion and butadiene selectivity were monitored over 110 hours and plotted against reaction time. The results are shown in FIG. 8.

Carbon balances were determined to give typically more than 95%.

### Example 3

In this example, the dehydrogenation reaction was investigated.

A feedstock of pure ethanol having a water content of less than 0.005 vol % was compared with a feedstock of 90 v/v % ethanol/H₂O. The temperature of the reaction was held at 300° C. The WHSV of the pure ethanol was 7.7 hr⁻¹, while the WHSV of the aqueous ethanol was 5.3 hr⁻¹.

The ethanol conversion and acetaldehyde selectivity of pure ethanol and aqueous ethanol are shown in FIG. 6a and FIG. 6b, respectively.

### Example 4

Different catalyst systems at different WHSV and time were investigated in this example.

The dual catalyst system illustrated in FIG. 4 was used and the results are shown in Table 2 below.

TABLE 2

| | Reactor 1 | Reactor 2 | EtOH | BD | WHSV |
|---|---|---|---|---|---|
| Entry | catalyst | catalyst | conversion (%) | Selectivity (%) | (hr⁻¹) |
| 1 | Cu/MCF | Zr/MCF | 99 | 73 | 1.5 |
| 2 | Cu/MCF | Zr/MCF | 99 | 70 | 1.5 |
| 3 | Cu/MCF | Zr/MCF | 99 | 72 | 1.5 |
| 4 | Cu/MCF | Zr/MCF | 96 | 69 | 3.7 |
| 5 | Cu/MCF | Zr/MCF | 85 | 71 | 6.5 |
| 6 | Cu/MCF | Zr/MCF | 92 | 71 | 1.5 |

The ethanol conversion and butadiene selectivity were calculated as an average over 20 hours.

In entries 1, 4 and 5, the Zr/MCF catalyst had an ICP loading of 2.0 mol %. In entry 2, the Zr/MCF catalyst had an ICP loading of 1.0 mol %. In entry 3, the Zr/MCF catalyst had an ICP loading of 3.0 mol %. In entries 1 to 4, pure ethanol having a water content of less than 0.005 vol % was used.

In entry 6, 4 mol % Cu and 2.0 mol % Zr were used as catalysts and wet ethanol was used (10 vol % H₂O).

The optimum temperatures for reactors 1 and 2 were 240° C. and 375° C., respectively

From entries 1, 2 and 3 in Table 2 above, it can be seen that the optimum ICP loading of the Zr/MCF catalyst was 2.0 mol %.

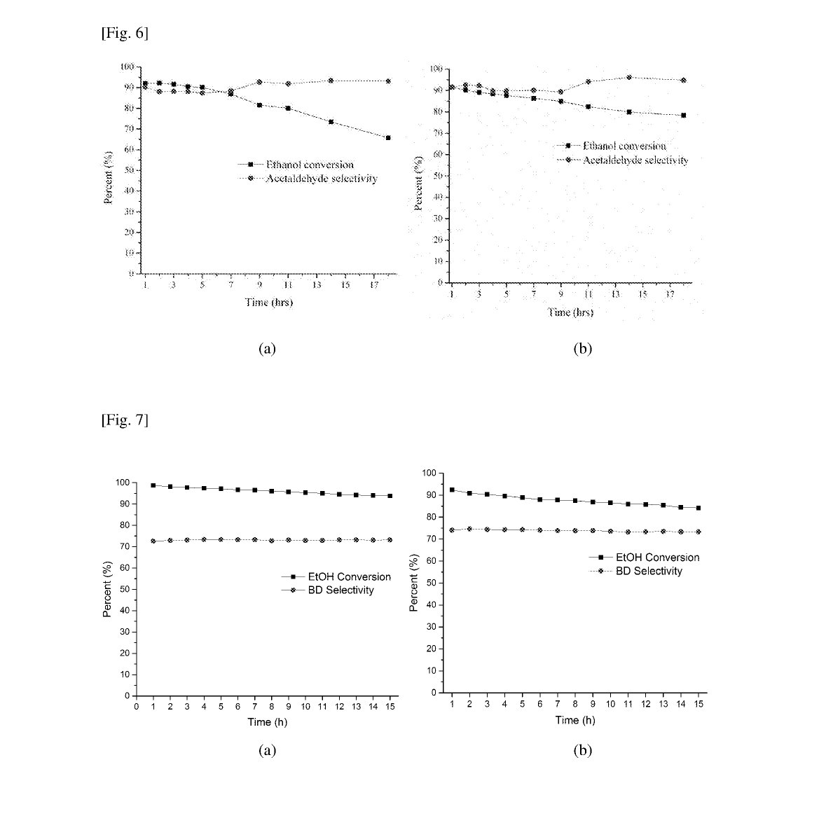

The ethanol conversion and butadiene selectivity of entry 1 were plotted against reaction time and is shown in FIG. 7a. The ethanol conversion and butadiene selectivity of entry 6 were plotted against reaction time and is shown in FIG. 7b. Upon comparison of FIG. 7a and FIG. 7b, it is shown that BD selectivity was comparable when pure ethanol or wet ethanol was used. However, it can be seen that ethanol conversion was slightly higher when pure ethanol was used as compared to when wet ethanol was used.

An average BD selectivity of 73% at an ethanol conversion of 96% was achieved in entry 1. BD productivity of 0.62 g_{BD}g_{cat}⁻¹ hr⁻¹ at a concentration of 1.7×10⁴ Vppm over a time-on-stream of 15 hours was also achieved. Accordingly, the catalytic performance of the present system surpassed all reported results known to the inventors, e.g. a BD productivity of at least 0.15 g_{BD}g_{cat}⁻¹ hr⁻¹ and a concentration of 1×10⁴ Vppm in the product stream reported in E. V. Makshina, W. Janssens, B. F. Sels and P. a. Jacobs, Catalysis Today, 2012, 198, 338-344.

### Example 5

The use of a different silica support for reactor 2 was investigated in this example. The dual catalyst system as illustrated in FIG. 9 was used.

The synthesis of this catalyst was identical to that for Zr/MCF in Example 1, except that three different silica supports (Merck, Davisil 60 Å grade 635 and 150 Å grade 645) were used in place of MCF.

The catalyst for reactor 1 remained as Cu/MCF.

The optimum temperatures of reactors 1 and 2 were 235° C. and 400° C., respectively.

Ethanol conversion and butadiene selectivity were averaged over 15 hrs at WHSV of 1.5 hr⁻¹. The results are shown in Table 3 below.

TABLE 3

| Zr/support | EtOH Conversion (%) | BD Selectivity (%) |
|---|---|---|
| Merck silica gel 60 | 91 | 65 |
| Davisil 60 Å grade 635 | 94 | 65 |
| Davisil 150 Å grade 645 | 90 | 64 |

### Example 6

The use of a binary catalyst for reactor 2 was investigated in this example. The dual catalyst system as illustrated in FIG. 10 was used.

The ethanol to butadiene reaction using M/Zr/MCF supported catalyst, where M is either cerium (Ce), copper (Cu), magnesium (Mg) or zinc (Zn) of various loadings, for the second reactor were carried out.

The catalyst for reactor 1 remained as Cu/MCF.

The optimum temperatures of reactors 1 and 2 were 235° C. and 400° C., respectively.

Conversion and selectivity values were obtained after 100 minutes into the reaction at WHSV of 1.5 hr⁻¹. The carbon balance was typically more than 95%. The results are shown in Table 4 below.

TABLE 4

| | EtOH Conversion (%) | BD Selectivity (%) |
|---|---|---|
| Mol ratio Zn/Zr/MCF | | |
| 0.01/2/98 | 99 | 75 |
| 0.05/2/98 | 99 | 76 |
| 0.1/2/98 | 98 | 70 |
| Mol ratio Mg/Zr/MCF | | |
| 0.01/2/98 | 99 | 74 |
| 0.05/2/98 | 98 | 75 |
| 0.1/2/98 | 99 | 71 |
| Mol ratio Cu/Zr/MCF | | |
| 0.01/2/98 | 88 | 71 |
| 0.05/2/98 | 99 | 71 |
| 0.1/2/98 | 99 | 72 |
| Mol ratio Ce/Zr/MCF | | |
| 0.01/2/98 | 97 | 73 |
| 0.05/2/98 | 98 | 73 |
| 0.1/2/98 | 99 | 72 |

### Example 7

### Preparation of a Binary Catalyst on MCF

20 mL of DI water was added to 1 g of MCF. The urea hydrolysis method was used for the preparation of the M/Zr/MCF catalyst. An appropriate amount of zirconium precursor (ZrOCl₂.8H₂O or ZrONO₃.xH₂O), M precursor (where M=cerium, copper, magnesium or zinc) precursor and urea in a mole ratio of 1 to 10 was added to the MCF/water mixture and was rapidly stirred. The resultant mixture was heated up to 90° C. and stirred for 6 hours. After cooling, the mixture was filtered, washed several times with water, and then dried under vacuum for 12 hours. The resulting powders were heated at 500° C. for 3 hours to obtain the final colourless M/Zr/MCF catalyst.

### Example 8

Scale-up experiments were investigated in this example.

The method of Example 6 was carried out at a larger scale, with the use of the dual catalyst system as illustrated in FIG. 10.

In reactor 1, 125 mg of Cu/MCF was used. In reactor 2, 375 mg of Zr/MCF was used.

At a temperature of 240° C. for reactor 1 and 385° C. for reactor 2, the average ethanol conversion was 84% and butadiene selectivity was 76% over 15 hrs, at a WHSV of 2.1 hr⁻¹. The nitrogen flow was 35 ml/min.

Two other M/Zr/MCF catalysts (M=Zn or Mg) were also used as catalyst in reactor 2.

The mol ratio of Zn/Zr/MCF and Mg/Zr/MCF are 0.05Zn/2Zr/98MCF and 0.05Mg/2Zr/98MCF respectively. Values of ethanol conversion and butadiene selectivity obtained are averaged over 15 hours. Results are shown in Table 5 below.

TABLE 5

| | | | EtOH | BD |
|---|---|---|---|---|
| | Reactor 2 | Reactor 1 | Conversion | Selectivity |
| Reactor 2 catalyst | temp. (° C.) | temp. (° C.) | (%) | (%) |
| Zr/MCF | 385 | 240 | 84 | 76 |
| Zn/Zr/MCF | 385 | 240 | 90 | 74 |
| Mg/Zr/MCF | 385 | 240 | 95 | 79 |

### INDUSTRIAL APPLICABILITY

The disclosed system and method may be useful to produce an optionally substituted olefin at high conversion yield and selectivity.

The first reaction zone is designed for efficient dehydrogenation reaction of the optionally substituted alcohol to an intermediate and the second reaction zone is designed for efficient coupling of the alcohol and intermediate and subsequent dehydration of the product to produce the optionally substituted olefin.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method for producing an optionally substituted olefin, comprising the steps of: dehydrogenating an optionally substituted alcohol in a first reaction zone comprising a first catalyst supported on a porous silica-based particle to form an optionally substituted carbonyl at a first set of reaction conditions; converting the optionally substituted alcohol and the optionally substituted carbonyl from the first reaction zone in a second reaction zone at a second set of reaction conditions that is different to the first set of reaction conditions and is selected to form the optionally substituted olefin, wherein the second reaction zone comprises a second catalyst supported on a porous silica-based particle.

2. The method of claim 1, wherein the first set of reaction conditions comprises controlling the alcohol/carbonyl molar ratio provided to the second reaction zone.

3. The method of claim 1, wherein the controlling step comprises fixing the temperature in the second reaction zone, while controlling the temperature in the first reaction zone.

4. The method of claim 1, wherein the first set of reaction conditions comprises conducting the dehydrogenating step at a temperature of between 100° C. to 400° C.

5. The method of claim 1, wherein the second set of reaction conditions comprises conducting the conversion step at a temperature of between 250° C. to 550° C.

6. The method of claim 1, wherein the first catalyst and the second catalyst are different metal catalysts composed of different metals.

7. The method of claim 1, wherein the first catalyst and the second catalyst are independently selected from the group consisting of a single metal catalyst, binary metal catalyst, ternary metal catalyst and metal oxide catalysts thereof.

8. The method of claim 7, wherein the metal or metal oxide of the first catalyst is selected from the group consisting of silver, gold, copper, cobalt, zinc, aluminum, magnesium, manganese, zirconium, tantalum, titanium, vanadium and their combinations thereof; and the metal or metal oxide of the second catalyst is selected from the group consisting of silver, gold, copper, zinc, aluminum, magnesium, zirconium, tantalum, titanium, vanadium and their combinations thereof.

9. The method of claim 7, wherein the first catalyst is copper supported on a mesoporous silica-based particle or wherein the second catalyst is zirconium supported on a mesoporous silica-based particle.

10. The method of claim 1, wherein the optionally substituted olefin is an optionally substituted diene; preferably
the optionally substituted olefin comprises 4 to 20 carbon atoms and the optionally substituted alcohol comprises 2 to 20 carbon atoms.

11. (canceled)

12. (canceled)

13. The method of claim 1, wherein the converting step comprises a step of coupling the optionally substituted alcohol with the optionally substituted carbonyl, and a step of dehydrating the coupled optionally substituted alcohol and optionally substituted carbonyl to form the optionally substituted olefin.

14. A system for producing an optionally substituted olefin, the system comprising:
a first reaction zone having a first catalyst for dehydrogenating an optionally substituted alcohol to form an optionally substituted carbonyl; and
a second reaction zone having a second catalyst for converting the optionally substituted alcohol
and the optionally substituted carbonyl from the first reaction zone to form the optionally substituted olefin;
wherein the first and second catalysts are supported on a porous silica-based particle.

15. The system of claim 14, wherein the silica-based particle is macroporous or mesoporous; preferably
the silica-based particle is mesoporous; or more preferably
the mesoporous silica-based particle is a mesocellular siliceous foam.

16. (canceled)

17. (canceled)

18. The system of claim 15, wherein the pore size of the mesocellular siliceous foam is in the range of 1 um to 100 um.

19. The system of claim 14, wherein the porous silica based particle has a surface area of at least 350 m²/g.

20. The system of 14, wherein the first catalyst and the second catalyst are independently selected from the group consisting of a single metal catalyst, binary metal catalyst, ternary metal catalyst and metal oxide catalysts thereof.

21. The system of claim 20, wherein the metal or metal oxide of the first catalyst is selected from the group consisting of silver, gold, copper, cobalt, zinc, aluminum, magnesium, manganese, zirconium, tantalum, titanium, vanadium and their combinations thereof; and the metal or metal oxide of the second catalyst is selected from the group consisting of silver, gold, copper, zinc, aluminum, magnesium, zirconium, tantalum, titanium, vanadium and their combinations thereof.

22. The system of claim 20, wherein the first catalyst is copper supported on the porous silica-based particle or
wherein the second catalyst is zirconium supported on the porous silica-based particle.

23. (canceled)

24. The system of claim 14, wherein the optionally substituted olefin is an optionally substituted diene; preferably
the optionally substituted olefin comprises 4 to 20 carbon atoms; or more preferably
the optionally substituted alcohol comprises 2 to 20 carbon atoms.

25. (canceled)

26. (canceled)
